(19) United States
(12) Patent Application Publication
(10) Pub. No.: US 2023/0404394 A1
(43) Pub. Date: Dec. 21, 2023

(54) Device And Method For Determining Glaucoma

(71) Applicant: THE CATHOLIC UNIVERSITY OF KOREA INDUSTRY-ACADEMIC COOPERATION FOUNDATION, Seoul (KR)

(72) Inventor: Yong Chan KIM, Seoul (KR)

(21) Appl. No.: 18/320,270

(22) Filed: May 19, 2023

(30) Foreign Application Priority Data
May 20, 2022 (KR) 10-2022-0061770

(51) Int. Cl. A61B 3/113; A61B 3/00; G16H 50/20

(52) U.S. Cl. CPC A61B 3/113; G16H 50/20; A61B 3/0091

(57) ABSTRACT

The disclosure relates to a glaucoma determination device and method. In particular, there may be provided a glaucoma determination device and method capable of determining the presence or absence of glaucoma from gaze tracking information. Specifically, there may be provided a glaucoma determination device and method capable of determining the presence or absence by determining the start time point and end time point of a gaze movement from gaze movement information and calculating area information about the gaze movement based thereupon.

### CROSS-REFERENCE TO RELATED APPLICATION

This application claims priority from Korean Patent Application No. 10-2022-0061770, filed on May 20, 2022, which is hereby incorporated by reference for all purposes as if fully set forth herein.

### BACKGROUND

### Field

The present embodiments provide a device and method for determining glaucoma.

### Description of Related Art

Glaucoma is an eye disease that causes glaucomatous changes in the optic nerve, resulting in impaired vision, which can progress over time and eventually lead to blindness if not treated properly. Also, unlike other eye diseases such as cataracts, once the optic nerve is damaged, it is impossible to restore optic nerve function through surgery, so it is necessary to diagnose glaucoma early and accurately. In the past, it was not easy to objectively detect optic nerve damage quickly. Due to recent research on glaucoma disease and the development of medical equipment technology, equipment that can detect optic nerve damage earlier than in the past has been developed and used in clinical practice. Measuring devices for glaucoma diagnosis are useful in ophthalmology centers, but have limitations in terms of portability and price, and over-satisfying consumer demand.

Eye Tracking is a technology that tracks eye movements by recognizing the pupil center and corneal reflex, and can be a scientific measurement tool that measures the point of gaze or the movement of the eye (pupil) to determine where to look. This eye tracking technology refers to the process of tracking the movement of the eye in conjunction with the gaze or head, and is a technology that acquires and analyzes information such as fixation, saccade, gaze pursuit, and gaze path, and is also used in the fields of user behavior analysis and user interface usability analysis.

Therefore, a need exists for a way to diagnose glaucoma more accurately without expensive measurement equipment by using eye tracking technology that is used in various fields.

### BRIEF SUMMARY

In the foregoing background, the present embodiments aim to provide a glaucoma determination device and method capable of determining the presence of glaucoma based on eye tracking information.

To achieve the foregoing objects, in an aspect, the present embodiments provide a glaucoma determination device comprising an information obtaining unit obtaining gaze tracking information including each gaze movement information measured using at least one target and time information regarding a start time point and an end time point determined from the gaze movement information, an information analysis unit calculating area information about a gaze movement with respect to position information about the target based on the gaze tracking information and generating area distribution information for each target using the area information, and a glaucoma determination unit determining a presence or absence of glaucoma using a classification model from the area distribution information for each target.

In another aspect, the present embodiments provide a glaucoma determination method comprising an information obtaining step obtaining gaze tracking information including each gaze movement information measured using at least one target and time information regarding a start time point and an end time point determined from the gaze movement information, an information analysis step calculating area information about a gaze movement with respect to position information about the target based on the gaze tracking information and generating area distribution information for each target using the area information, and a glaucoma determination step determining a presence or absence of glaucoma using a classification model from the area distribution information.

According to the present embodiments, there may be provided a glaucoma determination device and method capable of determining the presence of glaucoma based on eye tracking information.

### DESCRIPTION OF DRAWINGS

The above and other objects, features, and advantages of the disclosure will be more clearly understood from the following detailed description, taken in conjunction with the accompanying drawings, in which:

FIG. 1 is a view illustrating a configuration of a glaucoma determination device according to an embodiment of the disclosure;

FIG. 2 is a flowchart illustrating a determination operation of a glaucoma determination device according to an embodiment of the disclosure;

FIG. 3 is a view illustrating an operation of obtaining gaze movement information by a glaucoma determination device according to an embodiment of the disclosure;

FIG. 4 is a view illustrating an operation of obtaining time point information from gaze movement information by a glaucoma determination device according to an embodiment of the disclosure;

FIG. 5 is a view illustrating an operation of determining a start time point by a glaucoma determination device according to an embodiment of the disclosure;

FIG. 6 is a view illustrating an operation of determining an end time point by a glaucoma determination device according to an embodiment of the disclosure;

FIG. 7 is a view illustrating an operation of calculating area information by a glaucoma determination device according to an embodiment of the disclosure;

FIG. 8 is a view illustrating area distribution information generated by a glaucoma determination device according to an embodiment of the disclosure;

FIG. 9 is a view illustrating area distribution information for each target, generated by a glaucoma determination device according to an embodiment of the disclosure;

FIG. 10 is a view illustrating an operation of pre-processing area distribution information by a glaucoma determination device according to an embodiment of the disclosure;

FIG. 11 is a view illustrating a linear classification model of a glaucoma determination device according to an embodiment of the disclosure;

FIG. 12 is a view illustrating a machine learning algorithm-based classification model of a glaucoma determination device according to an embodiment of the disclosure;

FIG. 13 is a flowchart illustrating a glaucoma determination method according to an embodiment of the disclosure; and

FIG. 14 is a block diagram illustrating a glaucoma determination device according to an embodiment of the disclosure.

### DETAILED DESCRIPTION

The disclosure relates to a glaucoma determination device and method.

Hereinafter, embodiments of the disclosure are described in detail with reference to the accompanying drawings. The same or substantially the same reference denotations are used to refer to the same or substantially the same elements throughout the specification and the drawings. When determined to make the subject matter of the disclosure unclear, the detailed description of the known configurations or functions may be skipped.

Such denotations as “first,” “second,” “A,” “B,” “(a),” and “(b),” may be used in describing the components of the disclosure. These denotations are provided merely to distinguish a component from another, and the essence of the components is not limited by the denotations in light of order or sequence. When a component is described as “connected,” “coupled,” or “linked” to another component, the component may be directly connected or linked to the other component, but it should also be appreciated that other components may be “connected,” “coupled,” or “linked” between the components.

Hereinafter, embodiments of the disclosure are described in detail with reference to the accompanying drawings.

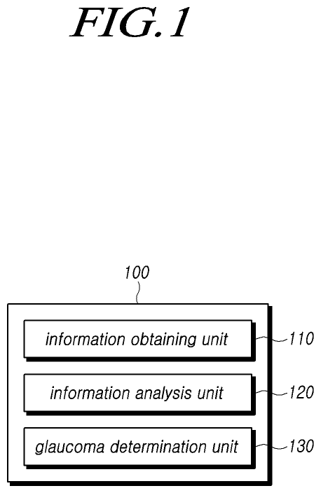

FIG. 1 is a view illustrating a configuration of a glaucoma determination device according to an embodiment of the disclosure.

Referring to FIG. 1, a glaucoma determination device 100 according to an embodiment of the disclosure comprises an information obtaining unit 110 obtaining gaze tracking information including each gaze movement information measured using at least one target and time information regarding a start time point and an end time point determined from the gaze movement information, an information analysis unit 120 calculating area information about a gaze movement with respect to position information about the target based on the gaze tracking information and generating area distribution information for each target using the area information, and a glaucoma determination unit 130 determining a presence or absence of glaucoma using a classification model from the area distribution information for each target.

The information obtaining unit 110 according to an embodiment may obtain gaze tracking information including gaze movement information and time point information. For example, the information obtaining unit 110 may obtain gaze tracking information including gaze movement information measured using at least one target and time point information about a start time point and an end time point determined from the gaze movement information. For example, the information obtaining unit 110 may obtain gaze movement information by measuring the eyeball moving to the target as the target is displayed while gazing at the fixation point. The information obtaining unit 110 may determine a time point of starting to move to the target meeting a preset condition according to a saccade included in the gaze movement information as the start time point. Further, the information obtaining unit 110 may determine a time point of reaching the target to start to gaze as the end time point. Specifically, the information obtaining unit 110 may determine a time point meeting all of the first conditions as the start time point with respect to at least one parameter calculated based on the time point when the saccade occurs. The information obtaining unit 110 may determine a time point meeting the second condition with respect to the distance to the target from the gaze movement information as the end time point.

The information analysis unit 120 according to an embodiment may analyze information by generating area information and area distribution information. For example, the information analysis unit 120 may calculate the area information about the gaze movement with respect to the position information about the target based on the gaze tracking information. The information analysis unit 120 may generate area distribution information for each target using the calculated area information. Here, the area distribution information may be generated by calculating area information for each subject based on gaze acquisition information obtained for a plurality of subjects and using the area information for each subject as coordinates. For example, the information analysis unit 120 may calculate the area information by calculating the difference from the gaze movement information with respect to the position information about the target in the period between the start time point and the end time point. Specifically, the information analysis unit 120 may divide the gaze movement information into X-axis movement information and Y-axis movement information over time. The information analysis unit 120 may calculate the difference from the X-axis movement information and the Y-axis movement information with respect to the position information about the target in the period between the start time point and the end time point, calculating each area information. Further, the information analysis unit 120 may display the calculated area information on the area distribution information with X-axis coordinates and Y-axis coordinates, respectively.

The glaucoma determination unit 130 according to an embodiment may determine whether there is glaucoma using a classification model from the area distribution information. For example, the glaucoma determination unit 130 may generate a classification model for determining glaucoma based on the area information included in the area distribution information. For example, the classification model may determine a threshold, as a reference for classification, from the area distribution information, and may determine that there is glaucoma when the area information corresponds to an area exceeding the threshold. Specifically, the classification model may determine a threshold by setting an arbitrary area based on the ratio of glaucoma subjects to normal subjects. If the area information displayed on the area distribution information exceeds the determined threshold, it may be determined that there is glaucoma. Also, the classification model may be a model that optimizes the threshold using an evaluation index of binary classification.

As another example, the glaucoma determination unit 130 may classify normal or glaucoma by a labeling method, and generate a classification model for determining glaucoma using a linear classification technique or a machine learning technique. For example, the classification model may be a machine learning algorithm-based classification model or a linear classification model learned using learning data generated by labeling a plurality of area information according to being normal or the presence or absence of glaucoma. Accordingly, the glaucoma determination unit 130 according to an embodiment may determine whether there is glaucoma by inputting the area distribution information to the classification model. In other words, each area information included in the area distribution information may be classified as normal or glaucoma.

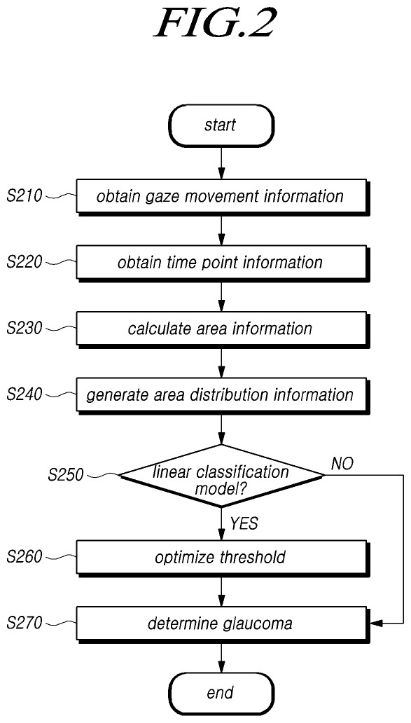

FIG. 2 is a flowchart illustrating a determination operation of a glaucoma determination device according to an embodiment of the disclosure.

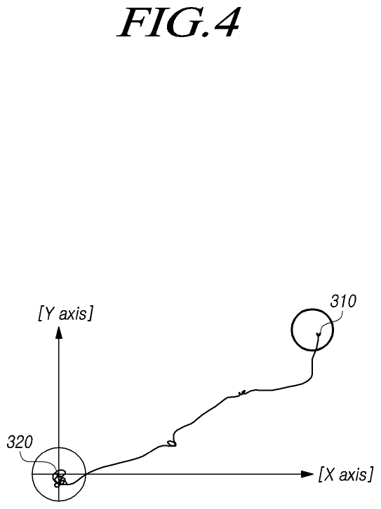

Referring to FIG. 2, the information obtaining unit 110 of the glaucoma determination device according to an embodiment of the disclosure may obtain gaze movement information (S210). For example, the information obtaining unit 110 may obtain each gaze movement information measured using at least one target. For example, the information obtaining unit 110 may obtain gaze movement information by measuring the gaze path of the eyeball moving to the target as the target is displayed while gazing at the fixation point. Specifically, the fixation point may be a point that the test subject should gaze at when the test starts, and the target may be a point that the test subject should move and gaze at after the fixation point disappears. Here, fixation is a state of the gaze path and may mean a state while the gaze path moves at a speed or acceleration equal to or less than a predetermined value. The gaze movement information is described below in detail with reference to FIG. 4.

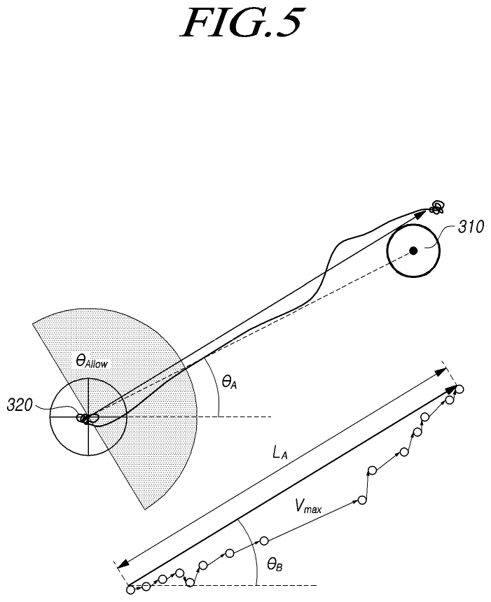

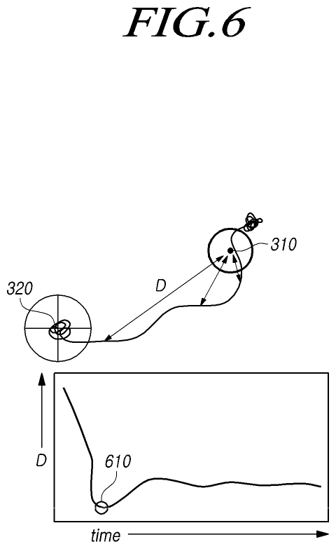

The information obtaining unit 110 of the glaucoma determination device according to an embodiment may obtain time point information (S220). For example, the information obtaining unit 110 may obtain time point information about the time point determined from the gaze movement information. For example, the information obtaining unit 110 may determine a start time point and an end time point using a start time point and end time point determination algorithm. Specifically, the information obtaining unit 110 may determine a time point of starting to move to the target meeting a preset condition according to a saccade included in the gaze movement information as the start time point and may determine a time point of reaching the target to start to gaze as the end time point. Here, saccade may mean a state immediately before the next fixation when the gaze path is not at the fixation. The start time and end time determination algorithm is described below in detail with reference to FIGS. 5 and 6.

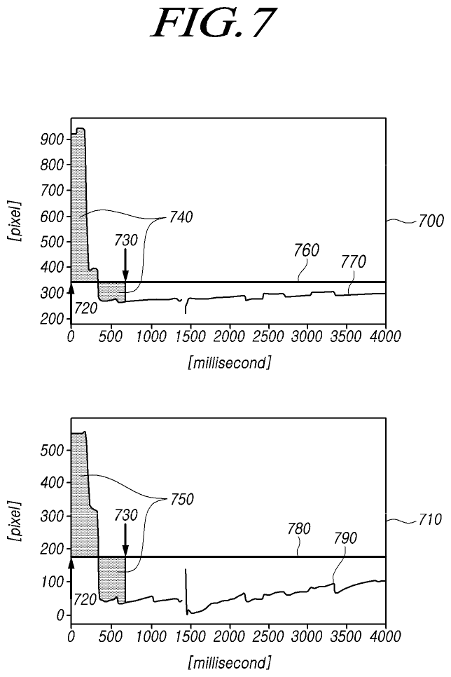

The information analysis unit 120 of the glaucoma determination device according to an embodiment may calculate area information (S230). For example, the information analysis unit 120 may calculate the area information about the gaze movement with respect to the position information about the target based on the gaze tracking information. For example, the information analysis unit 120 may divide the gaze movement information into X-axis movement information and Y-axis movement information over time. The information analysis unit 120 may calculate the difference from each axis movement information divided with respect to the position information about the target in the period between the start time point and the end time point, calculating each area information. The area information is described below in detail with reference to FIG. 7.

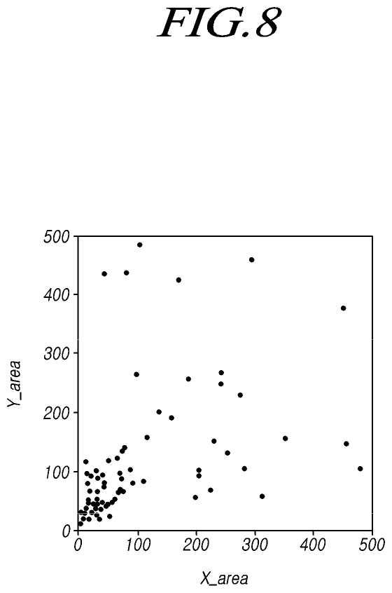

The information analysis unit 120 of the glaucoma determination device according to an embodiment may calculate area distribution information (S240). As an example, the information analysis unit 120 may generate area distribution information for each target using the area information calculated with respect to a specific target. For example, the information analysis unit 120 may display the area information calculated from each axis movement information, as an X-axis coordinate and a Y-axis coordinate, respectively, generating the area distribution information. Specifically, the information analysis unit 120 may generate the area distribution information by calculating area information for each subject based on gaze acquisition information obtained for a plurality of subjects and displaying the per-subject area information with the respective coordinates, generating the area distribution information. The generated area distribution information is described below in detail with reference to FIG. 8.

The glaucoma determination unit 130 of the glaucoma determination device according to an embodiment may determine a classification model (S250). For example, the glaucoma determination unit 130 may determine whether the classification model is a linear classification model or a classification model based on a machine learning algorithm. For example, the glaucoma determination unit 130 may generate a machine learning algorithm-based classification model or a linear classification model learned using learning data generated by labeling a plurality of area information according to being normal or the presence or absence of glaucoma. The glaucoma determination unit 130 may determine whether a classification model capable of determining the presence or absence of glaucoma using the area distribution information as an input is a linear classification model.

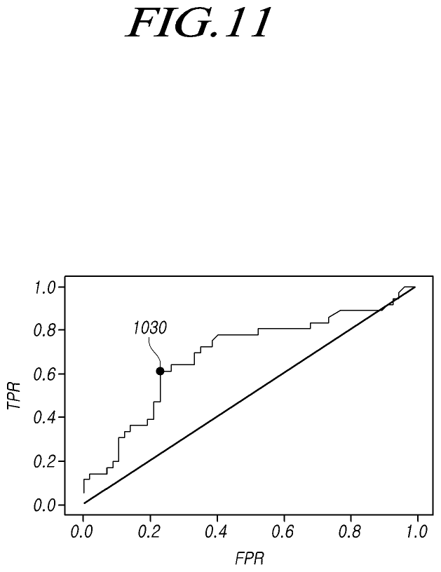

If the classification model is a linear classification model, the glaucoma determination unit 130 of the glaucoma determination device according to an embodiment may optimize the threshold serving as a classification reference (S260). For example, if the selected classification model is a linear classification model, the glaucoma determination unit 130 may optimize the threshold for distinguishing a normal area or glaucoma area. Here, the linear classification model may refer to a model to which a binary classification technique for binary classification is applied. For example, the glaucoma determination unit 130 may determine the threshold for each area information included in the area distribution information using the linear classification model and classify as normal or glaucoma based on the threshold. The threshold is described below in detail with reference to FIG. 11.

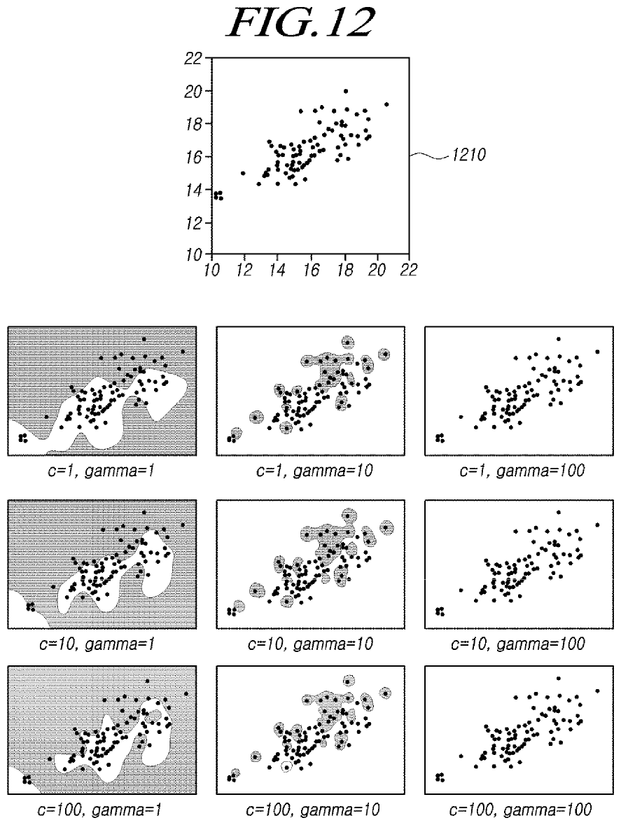

The glaucoma determination unit 130 of the glaucoma determination device according to an embodiment may determine whether there is glaucoma using a classification model from the area distribution information. As an example, the glaucoma determination unit 130 may determine the presence or absence of glaucoma using a linear classification model or machine learning algorithm-based classification model, from the area distribution information. For example, the glaucoma determination unit 130 may input the area distribution information to the classification model and classify each area information as normal or glaucoma. Specifically, the classification model may be a model that is generated based on a given data set using a classifier such as a support vector machine (SVM) and, if receiving new data, determine which item it belongs and classifies it. The glaucoma determination unit 130 may determine whether there is glaucoma using a classification model of a supervised learning approach based on a support vector machine. The classification model is described below with reference to FIGS. 11 and 12.

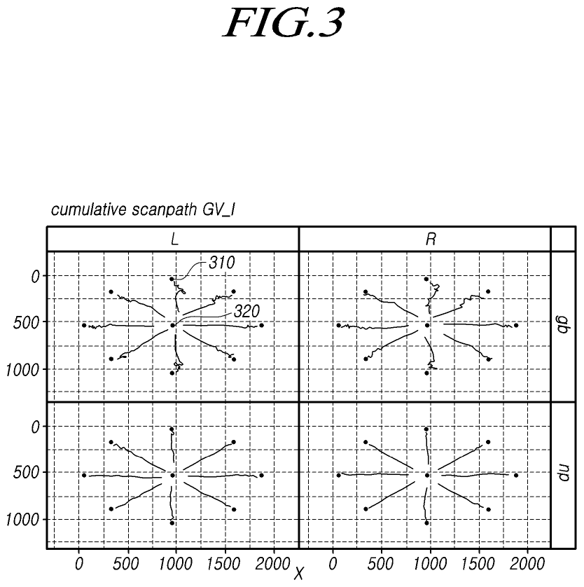

FIG. 3 is a view illustrating an operation of obtaining gaze movement information by a glaucoma determination device according to an embodiment of the disclosure.

Obtaining gaze movement information from at least one target by the glaucoma determination device according to an embodiment of the disclosure is described with reference to FIG. 3. For example, the information obtaining unit 110 of the glaucoma determination device 100 may obtain the eye movement of the subject according to the target 310 measured in real time through eye tracking as eye movement information. For example, the information obtaining unit 110 lets the subject gaze at the fixation point 320 which is distant forward and then implements a target 310 at a predetermined position and monitors the eyeball movement when gazing at the target to thereby obtain gaze movement information. During the eye tracking test, the target 310 may be implemented in at least one of eight directions: up, down, left, right, top left, bottom left, top right, and bottom right. Further, the gaze movement information may be information measured on each of a plurality of subjects, separately for the right and left eyes. Here, the subject may be a normal or glaucoma patient. The glaucoma patient may be a binocular glaucoma patient or a monocular glaucoma patient.

Specifically, in the gaze tracking test, whether to display the fixation point 320 and the target 310 may be changed as the test proceeds. Initially, the fixation point 320 is displayed, and the target 310 may be displayed simultaneously when the gaze point 320 disappears. The subject may gaze at the fixation point 320 and, after the target 310 is displayed, move the gaze toward the target 310. In this case, the time point T s when the subject starts to move the gaze toward the target 310 and the time point T_{E} when the gaze reaches the target 310 and starts to be fixed at the target 310 may vary from person to person. The time point Ts when the subject starts to move the gaze toward the target 310 may be the start time point of the gaze movement, and the time point T_{E} when the gaze reaches the target 310 and starts to be fixed at the target 310 may be the end time point of the gaze movement. However, the start time point may be determined according to whether the saccade included in the gaze movement information measured in the gaze tracking test meets a preset first condition. The end time point may be determined according to whether the second condition is met based on the distance to the target 310 from the gaze movement information. In other words, depending on whether the condition to be described later is met, even though the saccade occurs, it may not be determined as the start time point, and even though the vertical distance to the center of the target 310 is the minimum, it may not be determined as the end time point.

FIG. 4 is a view illustrating an operation of obtaining time point information from gaze movement information by a glaucoma determination device according to an embodiment of the disclosure.

Referring to FIG. 4, a glaucoma determination device according to an embodiment of the disclosure may obtain time point information about a start time point and an end time point determined from gaze movement information. For example, the information obtaining unit 110 of the glaucoma determination device 100 may divide the actual movement path of the eyeball included in the gaze movement information into X-axis movement information and Y-axis movement information over time. The start time point and the end time point may be determined using the start time point and end time point discrimination algorithm in each axis movement information. For example, the information obtaining unit 110 may determine a time point meeting all of the preset first conditions as the start time point based on the time point when a saccade occurs during the period from immediately after the fixation point 320 disappears and the target 310 appears to the end of the test. Further, the information obtaining unit 110 may determine a time point meeting the second condition with respect to the distance to the target 310 from the gaze movement information as the end time point.

FIG. 5 is a view illustrating an operation of determining a start time point by a glaucoma determination device according to an embodiment of the disclosure.

Referring to FIG. 5, the information obtaining unit 110 of the glaucoma determination device 100 according to an embodiment of the disclosure may determine a time point when a preset condition is met according to a saccade included in the gaze movement information as a start time point. As an example, the information obtaining unit 110 may determine a time point meeting all of the preset first conditions as the start time point with respect to at least one parameter calculated based on the time point when the saccade occurs. For example, the information obtaining unit 110 may determine the time point when a saccade meeting all of the first conditions occurs as the start time point during the period from immediately after the fixation point 320 disappears and the target 310 appears to the end of the test. Here, the eye movement information may be composed of a plurality of points measured by the eye tracking device. The saccade may be a vector connecting the start point and the end point of gaze movement information between one fixation and the next fixation. Also, the saccade may be configured as a set including a plurality of pieces of gaze movement information as its internal components.

Specifically, the information obtaining unit 110 may calculate the angle, length, and speed parameters based on the time point when the saccade appears and the time point when the saccade ends, and determine the time point when the calculated parameters all meet the first conditions as the start time point. The first condition for the angle parameter may be set as whether the difference between the angle θ_{A} of the line from the fixation point 320 to the target 310 and the angle θ_{B} from the start point to the end point of the saccade is within a predetermined range (e.g., 180 deg). The first condition for the length parameter may be set as whether the length LA from the start point to the end point of the saccade is equal to or greater than a predetermined value (e.g., 6 deg) based on the angle of the eyeball. Also, the first condition for the speed parameter may be set as whether the maximum speed of the saccade is greater than or equal to a predetermined value (e.g., 30 deg/sec).

As another example, when there are several saccades during one test, the information obtaining unit 110 may determine only the time point when the first saccade meeting the first condition occurs as the start time point.

FIG. 6 is a view illustrating an operation of determining an end time point by a glaucoma determination device according to an embodiment of the disclosure.

Referring to FIG. 6, the information obtaining unit 110 of the glaucoma determination device 100 according to an embodiment of the disclosure may determine a time point when a preset condition is met based on the distance to the target from the gaze movement information as the end time point. As an example, the information obtaining unit 110 may determine a time point meeting the preset second condition with respect to the distance to the target from the gaze movement information as the end time point. The second condition may be set as whether the vertical distance from the gaze path to the center of the target 310 is minimum. For example, the information obtaining unit 110 may determine a time point 610 when the vertical distance from the gaze path to the center of the target 310 is minimized during the period from immediately after the fixation point 320 disappears and the target 310 appears to the end of the inspection, as the end time point.

Specifically, the information obtaining unit 110 may calculate the vertical distance D from the position Pₜ(Xₜ, Yₜ) on the gaze path to the center (X_{c}, Y_{c}) of the target 310 at a certain time t from start to end of the test. The vertical distance D may be expressed as Equation 1.

D={square root over (X_{c}−Xₜ)²+(Y_{c}−Yₜ)²)} [Equation 1]

However, as another example, when it is determined that there are a plurality of time points 610 when the vertical distance is minimized, the information obtaining unit 110 may determine the time point when the vertical distance is minimized for the first time as the end time point.

FIG. 7 is a view illustrating an operation of calculating area information by a glaucoma determination device according to an embodiment of the disclosure.

Referring to FIG. 7, an X-axis movement path graph 700 and a Y-axis movement path graph 710 in which the information analysis unit 120 of the glaucoma determination device 100 calculates area information may be described. For example, the information analysis unit 120 may calculate the area information about the gaze movement, for each axis, with respect to the position information about the target based on the gaze tracking information. For example, the information analysis unit 120 may divide the gaze movement information into the X-axis movement information 770 and the Y-axis movement information 790 over time, and calculate the area information by calculating the difference between the position information about the target and each divided axis movement information in the period between the start time point 720 and the end time point 730. Specifically, the information analysis unit 120 may calculate the difference between the X-axis position information 760 and X-axis movement information 770 about the target in the period between the start time 720 and the end time 730 on the X-axis movement path graph 700, and calculate the absolute value of the calculated difference value as X-axis area information 740. Further, the information analysis unit 120 may calculate the difference between the Y-axis position information 780 and Y-axis movement information 790 about the target in the period between the start time 720 and the end time 730 on the Y-axis movement path graph 710, and calculate the absolute value of the calculated difference value as Y-axis area information 750.

FIG. 8 is a view illustrating area distribution information generated by a glaucoma determination device according to an embodiment of the disclosure.

Referring to FIG. 8, the information analysis unit 120 of the glaucoma determination device 100 may generate area distribution information using each calculated area information. For example, the information analysis unit 120 may calculate area information for each subject based on gaze acquisition information obtained based on a specific target for a plurality of subjects. Further, the information analysis unit 120 may generate area distribution information about a specific target using the calculated area information for each subject as each coordinate. Here, the area distribution information may be represented as an area distribution graph. For example, the information analysis unit 120 may generate an area distribution graph of a specific target using X-axis area information and Y-axis area information about the subject as X-axis coordinate and Y-axis coordinate, respectively. Then, the glaucoma determination unit 130 may determine which one of the normal area or the glaucoma area in the area distribution graph the coordinates for the area information about the subject is positioned in. When it is determined that the area information about the subject is positioned in the glaucoma area, the glaucoma determination unit 130 may determine the subject as glaucoma.

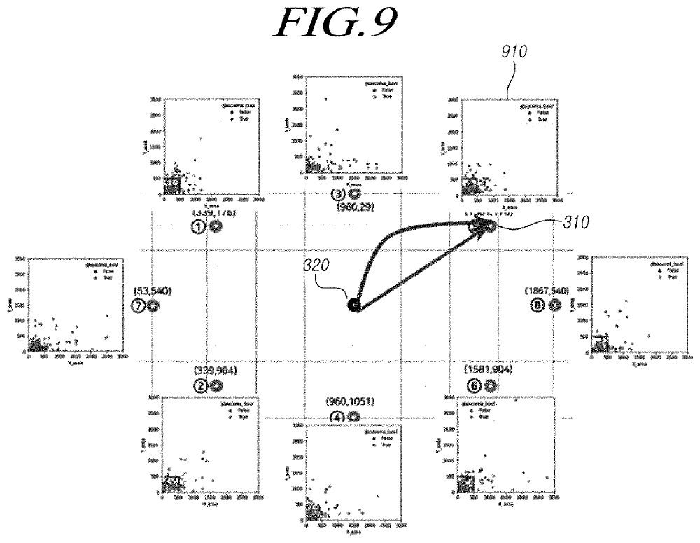

FIG. 9 is a view illustrating area distribution information for each target, generated by a glaucoma determination device according to an embodiment of the disclosure.

Referring to FIG. 9, the information analysis unit 120 of the glaucoma determination device 100 may generate area distribution information for each target using area information calculated based on at least one target. For example, the information obtaining unit 110 may allow a plurality of subjects to see the fixation point 320 distant forward, implement a specific target 310 at a predetermined position, and obtain gaze acquisition information based thereupon. The information analysis unit 120 may generate the area distribution information 910 about the specific target 310 by calculating the area information for each subject based on the plurality of gaze acquisition information. If the target 310 is implemented in at least one position among eight directions: up, down, left, right, top left, bottom left, top right, and bottom right, area distribution information 910 may be generated for each implemented target. Specifically, the information analysis unit 120 may select one of the most important specific targets and use the area distribution information about the specific target as a reference for determining glaucoma. Alternatively, the information analysis unit 120 may combine area distribution information about all targets implemented in the eight directions and use the area distribution information as a reference for determining glaucoma. The information analysis unit 120 may count when any one determination result among the determination results of area distribution information for all the targets is classified as normal and when classified as glaucoma and determine the presence or absence of glaucoma based on the sum of the counts. However, this is merely an example of determining in combination and the disclosure is not limited thereto.

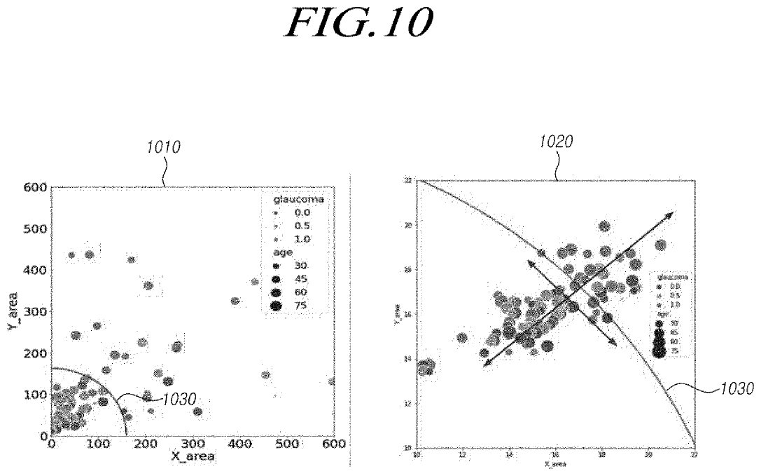

FIG. 10 is a view illustrating an operation of pre-processing area distribution information by a glaucoma determination device according to an embodiment of the disclosure.

Referring to FIG. 10, the information analysis unit 120 of the glaucoma determination device 100 may pre-process the generated area distribution information to determine glaucoma with higher accuracy. For example, the information analysis unit 120 may generate area distribution information 1010 with parameters added. For example, the information analysis unit 120 may adjust the position by assigning a weight to each area information included in the area distribution information according to importance. Specifically, the information analysis unit 120 may adjust the position in the area distribution graph by adding at least one of the subject's age information and glaucoma diagnosis information as a parameter and assigning a weight to each parameter. Further, the parameter may be set as constitution information, gender information, or the like, in addition to the subject's age information and glaucoma diagnosis information.

As another example, the information analysis unit 120 may generate area distribution information 1020 by scaling by a correction factor. For example, the information analysis unit 120 may generate the adjusted area distribution information by scaling each area information included in the area distribution information by a correction factor. In other words, the information analysis unit 120 may scale the area information across the threshold 1030, which is a reference for classification, in the area distribution information to be distributed in a larger range. Specifically, the information analysis unit 120 may calculate a distance for each area information included in the area distribution information, and scale by applying a correction factor so that the calculated distance may be accurately classified based on the threshold 1030. The correction factor may be a multiplier, offset, or function used to scale, transition, or otherwise adjust each area information.

FIG. 11 is a view illustrating a linear classification model of a glaucoma determination device according to an embodiment of the disclosure.

An example in which the glaucoma determination unit 130 of the glaucoma determination device 100 determines a threshold 1030 for determining normal or glaucoma through a linear classification model is described with reference to FIG. 11. For example, the glaucoma determination unit 130 may determine a threshold 1030 that is a reference for classification in the area distribution graph and may determine it as glaucoma when the area exceeds the threshold 1030. Here, the shape of the threshold 1030 may have several linear or nonlinear shapes such as a circle, an oval, and a square. For example, the glaucoma determination unit 130 may determine the optimal threshold 1030 while increasing the threshold 1030 from 0 in the area distribution graph. Further, the glaucoma determination unit 130 may determine that the subject is normal if the distance calculated based on the area information included in the area distribution graph is the threshold 1030 or less and, if the distance is the threshold 1030 or more, determine that the subject has glaucoma. The distance calculated based on the area information may be expressed as Equation 2.

distance=\sqrt{(Xₐᵣₑₐ)²+(Yₐᵣₑₐ)²} [Equation 2]

As another example, the glaucoma determination unit 130 may set an arbitrary area in the area distribution graph, divide the area into an area classified as normal and an area classified as glaucoma, and determine the threshold 1030 using the proportion of glaucoma subjects obtained based on the normal area and the glaucoma area. When the coordinates of the area information in the area distribution graph correspond to an area exceeding the threshold 1030, the glaucoma determination unit 130 may determine that there is glaucoma. Specifically, the threshold 1030 may be determined based on a section in which the ratio of subjects classified as glaucoma to all subjects is 95%. However, 95% is an example, and the disclosure is not limited thereto.

As another example, the glaucoma determination unit 130 may determine the threshold 1030 that is the reference for classification using the evaluation criterion of the binary classification, and may determine it as glaucoma when the area exceeds the threshold 1030. Here, the evaluation criteria for the binary classification may be various indicators such as receiver operating characteristic (ROC)-curve, area under curve (AUC), accuracy, recall, specificity, false alarm rate, precision, F1 score, and confusion matrix. These indicators may be calculated using true positive (TP), true negative (TN), false positive (FP), and false negative (FN).

For another example, the glaucoma determination unit 130 may cluster the area information into respective groups: normal; and glaucoma, using the evaluation criteria of the binary classification. Then, the boundary value between the clustered groups may be determined as the threshold 1030. Specifically, the glaucoma determination unit 130 may analyze at least one area information included in the area distribution graph to calculate their respective precisions (e.g., the ratio of the area information corresponding to the subject with actual glaucoma to the area information determined as glaucoma) and recalls (e.g., the ratio of the area information determined as glaucoma through the classification model to the area information corresponding to the subject with actual glaucoma). The glaucoma determination unit 130 may calculate an F1 score that indicates classification performance for one or more area information using precision and reproducibility. However, without limitations thereto, the glaucoma determination unit 130 may calculate a false positive rate (FPR) (e.g., a ratio of area information determined to be glaucoma through the classification module to area information corresponding to actual normal). Accordingly, the glaucoma determination unit 130 may determine the optimal threshold 1030 using the binary classification evaluation criterion.

FIG. 12 is a view illustrating a machine learning algorithm-based classification model of a glaucoma determination device according to an embodiment of the disclosure.

Referring to FIG. 12, the glaucoma determination unit 130 of the glaucoma determination device 100 may determine whether there is glaucoma through a classification model based on a machine learning algorithm. For example, the glaucoma determination unit 130 may determine whether there is glaucoma from the area distribution information 1210 through a linear classification model or a classification model based on a machine learning algorithm trained with learning data labeled according to normal or glaucoma. For example, the glaucoma determination unit 130 may prepare learning data for training and testing, select an appropriate machine learning algorithm, and select, and train with, characteristic values to be used in the classification model. Specifically, the glaucoma determination unit 130 may determine whether there is glaucoma using a classification model of a supervised learning method based on a support vector machine (SVM). Here, the classification model may be a model that is generated based on a given data set using a classifier such as a support vector machine (SVM) and, if receiving new data, determine which item it belongs and classifies it. The support vector machine is a supervised learning method, but may be applied to both classification and regression problems. Further, the support vector machine may generate an optimal hyperplane in an iterative manner of maximizing a margin, thereby maximizing data intervals of different groups having closest distances. Accordingly, when the classification model is based on a support vector machine, the area information about the subject included in the area distribution information 1210 may be classified as normal or glaucoma using linear and Gaussian kernels. However, the machine learning algorithm is not limited to a support vector machine.

A glaucoma determination method that may be performed by the glaucoma determination device described above in connection with FIGS. 1 to 12 is described below.

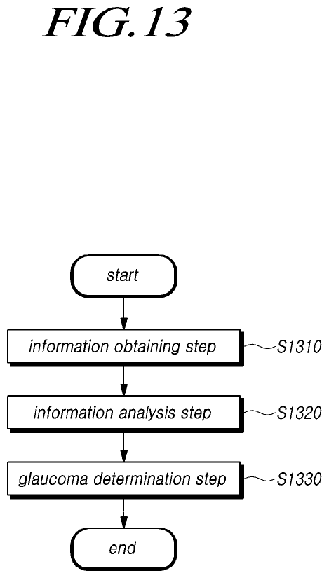

FIG. 13 is a flowchart illustrating a glaucoma determination method according to an embodiment of the disclosure.

Referring to FIG. 13, a glaucoma determination method according to an embodiment of the disclosure may include an information obtaining step S1310 of obtaining gaze tracking information including gaze movement information and time point information. For example, the glaucoma determination device may obtain gaze tracking information including gaze movement information measured using at least one target and time point information about a start time point and an end time point determined from the gaze movement information. For example, the glaucoma determination device may obtain gaze movement information by measuring the eyeball moving to the target as the target is displayed while gazing at the fixation point. Further, the glaucoma determination device may determine a time point of starting to move to the target meeting a preset condition according to a saccade included in the gaze movement information as the start time point and may determine a time point of reaching the target to start to gaze as the end time point. Specifically, the glaucoma determination device may determine a time point meeting all of the first conditions as the start time point with respect to at least one parameter calculated based on the time point when the saccade occurs. The glaucoma determination device may determine a time point meeting the second condition with respect to the distance to the target from the gaze movement information as the end time point.

The glaucoma determination method according to an embodiment may include an information analysis step S1320 of generating area information and area distribution information and analyzing the information. For example, the glaucoma determination device may calculate the area information about the gaze movement with respect to the position information about the target based on the gaze tracking information. The glaucoma determination device may generate area distribution information for each target using the calculated area information. Here, the area distribution information may be generated by calculating area information for each subject based on gaze acquisition information obtained for a plurality of subjects and using the area information for each subject as coordinates. For example, the glaucoma determination device may calculate the area information from the gaze movement information with respect to the position information about the target in the period between the start time point and the end time point. Specifically, the glaucoma determination device may divide the gaze movement information into X-axis movement information and Y-axis movement information over time. The glaucoma determination device may calculate area information by calculating the difference with respect to position information about the target from each axis movement information divided in the period between the start time point and the end time point. Further, the glaucoma determination device may display the area information calculated from each axis movement information, as the X-axis coordinate and Y-axis coordinate, respectively, on the area distribution information.

The glaucoma determination method according to an embodiment may include a glaucoma determination step S1330 of determining a presence or absence of glaucoma using a classification model from the area distribution information. For example, the glaucoma determination device may generate a classification model for determining glaucoma based on the area information included in the area distribution information. For example, the classification model may determine a threshold, as a reference for classification, from the area distribution information, and may determine that there is glaucoma when the area information corresponds to an area exceeding the threshold. Specifically, the classification model may determine a threshold by setting an arbitrary area based on the ratio of glaucoma subjects to normal subjects. If the area information displayed on the area distribution information exceeds the determined threshold value, it may be determined that there is glaucoma. Also, the classification model may be a model that optimizes the threshold value using an evaluation index of binary classification.

As another example, the glaucoma determination device may classify normal or glaucoma by a labeling method, and generate a classification model for determining glaucoma using a linear classification technique or a machine learning technique. For example, the classification model may be a machine learning algorithm-based classification model or a linear classification model learned using learning data generated by labeling a plurality of area information according to being normal or the presence or absence of glaucoma. Accordingly, the glaucoma determination device according to an embodiment may determine whether there is glaucoma by inputting the area distribution information to the classification model. In other words, each area information included in the area distribution information may be classified as normal or glaucoma.

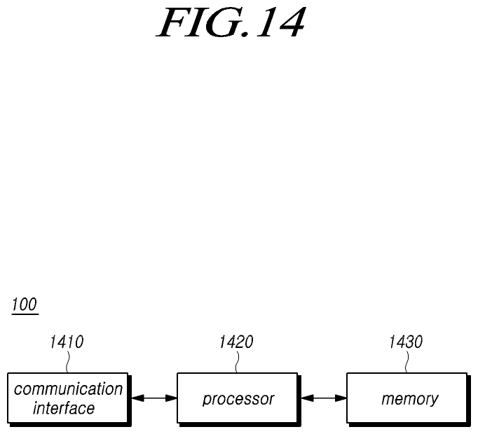

FIG. 14 is a block diagram illustrating a glaucoma determination device according to an embodiment of the disclosure.

Referring to FIG. 14, a glaucoma determination device 100 according to an embodiment includes a communication interface 1410 and a processor 1420. The glaucoma determination device 100 may further include a memory 1430. Each component, communication interface 1410, processor 1420, and memory 1430 may be connected to each other through a communication bus. For example, the communication bus may include a circuit for connecting the components with one another and transferring communications (e.g., control messages and/or data) between the components.

The communication interface 1410 may obtain measurement information in which a result of the gaze tracking test is recorded. Further, the communication interface 1410 may perform communication with an external device through wireless communication or wired communication.

The processor 1420 may perform at least one method described above in connection with FIGS. 1 to 13 or an algorithm corresponding to at least one method. The processor 1420 may be a data processing device implemented in hardware having a circuit having a physical structure for executing desired operations. For example, the desired operations may include code or instructions included in a program. For example, the data processing unit implemented in hardware may include a microprocessor, a central processing unit, a processor core, a multi-core processor, a multiprocessor, a neural processing unit (NPU), an application-specific integrated circuit (ASIC), or a field programmable gate array (FPGA).

Further, the processor 1420 may execute the program and may control the glaucoma determination device 100. The program code executed by the processor 1420 may be stored in an internal memory or an external memory, i.e., the memory 1430. For example, the memory 1430 may store an image in which a result of eye tracking test obtained through the communication interface 1410 is recorded. Further, the memory 1430 may store various information generated during processing by the processor 1420 and output information extracted by the processor 1420. The output information may be information analyzed from gaze tracking information and may be information stored in a preset template form. The memory 1430 may store a result of determining whether there is glaucoma. The determination result may be obtained from the glaucoma determination device 100 or may be obtained from an external device. Further, the memory 1430 may store various data and programs. The memory 1430 may include a volatile memory or a non-volatile memory. The memory 1430 may include a mass storage medium, such as a hard disk and the like, and may store various data.

Although it is described above that all of the components are combined into one or are operated in combination, embodiments of the disclosure are not limited thereto. One or more of the components may be selectively combined and operated as long as it falls within the scope of the objects of the disclosure. Further, although all of the components may be implemented in their respective independent hardware components, all or some of the components may be selectively combined to be implemented in a computer program with program modules performing all or some of the functions combined in one or more hardware components and recorded in a computer-readable medium. The computer-readable medium may include programming commands, data files, or data structures, alone or in combinations thereof. The programming commands recorded in the medium may be specially designed and configured for the disclosure or may be known and available to one of ordinary skill in the computer software-related art. Examples of the computer readable recording medium may include, but is not limited to, magnetic media, such as hard disks, floppy disks or magnetic tapes, optical media, such as CD-ROMs or DVDs, magneto-optical media, such as floptical disks, memories, such as ROMs, RAMS, or flash memories, or other hardware devices specially configured to retain and execute programming commands. Examples of the programming commands may include, but are not limited to, high-level language codes executable by a computer using, e.g., an interpreter, as well as machine language codes as created by a compiler. The above-described hardware devices may be configured to operate as one or more software modules to perform operations according to an embodiment of the disclosure, or the software modules may be configured to operate as one or more hardware modules to perform the operations.

When an element “comprises,” “includes,” or “has” another element, the element may further include, but rather than excluding, the other element, and the terms “comprise,” “include,” and “have” should be appreciated as not excluding the possibility of presence or adding one or more features, numbers, steps, operations, elements, parts, or combinations thereof. All the scientific and technical terms as used herein may be the same in meaning as those commonly appreciated by a skilled artisan in the art unless defined otherwise. It will be further understood that terms, such as those defined dictionaries, should be interpreted as having a meaning that is consistent with their meaning in the context of the relevant art and will not be interpreted in an idealized or overly formal sense unless expressly so defined herein.

The above-described embodiments are merely examples, and it will be appreciated by one of ordinary skill in the art various changes may be made thereto without departing from the scope of the disclosure. Accordingly, the embodiments set forth herein are provided for illustrative purposes, but not to limit the scope of the disclosure, and should be appreciated that the scope of the disclosure is not limited by the embodiments. The scope of the disclosure should be construed by the following claims, and all technical spirits within equivalents thereof should be interpreted to belong to the scope of the disclosure.

What is claimed is:

1. A glaucoma determination device, comprising:
an information obtaining unit obtaining gaze tracking information including each gaze movement information measured using at least one target and time information regarding a start time point and an end time point determined from the gaze movement information;
an information analysis unit calculating area information about a gaze movement with respect to position information about the target based on the gaze tracking information and generating area distribution information for each target using the area information; and
a glaucoma determination unit determining a presence or absence of glaucoma using a classification model from the area distribution information for each target.

2. The glaucoma determination device of claim 1, wherein the information obtaining unit determines that a time point of starting to move to the target meeting a preset condition according to a saccade included in the gaze movement information is the start time point, and a time point of starting to reach the target to gaze is the end time point.

3. The glaucoma determination device of claim 2, wherein the information obtaining unit determines that a time point of meeting all first conditions with respect to at least one parameter calculated based on a time point when the saccade occurs is the start time point, and a time point of meeting a second condition with respect to a distance to the target from the gaze movement information is the end time point.

4. The glaucoma determination device of claim 1, wherein the information analysis unit calculates the area information from the gaze movement information with respect to position information about the target in a period between the start time point and the end time point.

5. The glaucoma determination device of claim 1, wherein the area distribution information is generated by calculating area information for each subject based on gaze acquisition information obtained for a plurality of subjects and using the area information for each subject as coordinates.

6. The glaucoma determination device of claim 1, wherein the classification model determines a threshold, as a reference for classification, from the area distribution information and, if the area information corresponds to an area exceeding the threshold, determines that there is glaucoma.

7. The glaucoma determination device of claim 6, wherein the classification model optimizes the threshold using an evaluation index of binary classification.

8. The glaucoma determination device of claim 1, wherein the classification model is a machine learning algorithm-based classification model or a linear classification model learned using learning data generated by labeling a plurality of area information according to being normal or the presence or absence of glaucoma.

9. A glaucoma determination method, comprising:
an information obtaining step obtaining gaze tracking information including each gaze movement information measured using at least one target and time information regarding a start time point and an end time point determined from the gaze movement information;
an information analysis step calculating area information about a gaze movement with respect to position information about the target based on the gaze tracking information and generating area distribution information for each target using the area information; and
a glaucoma determination step determining a presence or absence of glaucoma using a classification model from the area distribution information.

10. The glaucoma determination method of claim 9, wherein the information obtaining step determines that a time point of starting to move to the target meeting a preset condition according to a saccade included in the gaze movement information is the start time point, and a time point of starting to reach the target to gaze is the end time point.

11. The glaucoma determination method of claim 10, wherein the information obtaining step determines that a time point of meeting all first conditions with respect to at least one parameter calculated based on a time point when the saccade occurs is the start time point, and a time point of meeting a second condition with respect to a distance to the target from the gaze movement information is the end time point.

12. The glaucoma determination method of claim 9, wherein the information analysis step calculates the area information from the gaze movement information with respect to position information about the target in a period between the start time point and the end time point.

13. The glaucoma determination method of claim 9, wherein the area distribution information is generated by calculating area information for each subject based on gaze acquisition information obtained for a plurality of subjects and using the area information for each subject as coordinates.

14. The glaucoma determination method of claim 9, wherein the classification model determines a threshold, as a reference for classification, from the area distribution information and, if the area information corresponds to an area exceeding the threshold, determines that there is glaucoma.

15. The glaucoma determination method of claim 9, wherein the classification model is a machine learning algorithm-based classification model or a linear classification model learned using learning data generated by labeling a plurality of area information according to being normal or the presence or absence of glaucoma.
